(19) United States
(12) Patent Application Publication
(10) Pub. No.: US 2006/0269978 A1
(43) Pub. Date: Nov. 30, 2006

(54) Method and device for monitoring platelet function

(76) Inventors: William S. Haworth, Columbia Heights, MN (US); Vincent J. Swenson, Plymouth, MN (US)

Correspondence Address: POPOVICH, WILES & O'CONNELL, PA;650 THIRD AVENUE SOUTH, SUITE 600, MINNEAPOLIS, MN, 55402, US

(21) Appl. No.: 11/409,735

(22) Filed: Apr. 24, 2006

Related U.S. Application Data
(60) Provisional application No. 60/674,501, filed on Apr. 25, 2005.

(51) Int. Cl. C12Q 1/56; C12M 1/34; C12M 3/00

(52) U.S. Cl. 435/13; 435/287.1

(57) ABSTRACT

A method of monitoring platelet function comprising: passing blood removed from a mammal through a passageway comprising a spring to contact the spring to generate a platelet mass in the passageway; and monitoring the flow or composition of the blood in the passageway to detect formation of the platelet mass. Devices and articles for performing the methods are also described.

This application claims the benefit of U.S. Provisional Application No. 60/674,501, filed Apr. 25, 2005, entitled “Method and Device for Monitoring Platelet Function,” the contents of which are hereby incorporated by reference herein.

### FIELD OF THE INVENTION

The present invention relates to a method and device for monitoring platelet function.

### BACKGROUND OF THE INVENTION

Platelets are anucleated cells that are the primary cells responsible for stopping bleeding. Blood platelets are approximately 3 microns in size and circulate in the blood stream as disc-shaped cells that upon activation by either tissue injury or exposure to a foreign material undergo physiological changes that lead to aggregate formation at the site of injury or foreign material. Blood platelets circulate at approximately 250,000 to 350,000 platelets per microliter of whole blood. Upon activation, platelets change shape from a disc to a sphere and form pseudopodia elongations.

The normal platelet response to initiate cessation of bleeding is to undergo a shape change, attach to the surface, and release intraplatelet components that act to provide an autocatalytic recruitment of more platelets. With the recruitment of additional platelets, a platelet plug or aggregate mass forms. The aggregate mass evolves from a single platelet of only 3 microns in size to a mass on the order of millimeters in size. The platelet mass additionally recruits and participates with the plasma coagulation proteins. The plasma coagulation proteins undergo a cascade of events involving 13 enzymes and cofactors, which leads to the activation of plasma fibrin to form a fibrin clot.

It is useful here to briefly summarize the biochemical events of hemostasis (the cessation of bleeding). Normal intact endothelium does not initiate or support platelet adhesion (although in certain vascular diseases platelets may adhere to intact endothelium). Vascular injury, however, exposes the endothelial surface and underlying collagen. Following vascular injury, platelets attach to adhesive proteins such as collagen via specific glycoproteins on the platelet surface. This adhesion is followed or accompanied by platelet activation, where platelets undergo a shape change from a disc shape to a spherical shape with extended pseudopodia. At this time, the platelet release reaction also occurs. The platelets release biologically active compounds stored in the cytoplasmic bodies that stimulate platelet activation or are otherwise involved in clotting reactions. These include ADP, serotonin, thromboxaneA₂, and von Willebrand factor. Thromboxane A₂ is a potent inducer of platelet secretion and aggregation. It is formed by the enzyme cyclooxgenase, which is inhibited by aspirin, among other drugs.

Following activation, glycoprotein IIb and ma (GPIIbIIIa) receptors on the surface of the platelets undergo a conformational change from a relatively inactive conformation to an activated form. GPIIbIIIa receptors mediate the adhesion of more platelets by adhering to the circulating plasma protein fibrinogen, which serves as a bridging ligand between platelets. The adhesion and aggregation of platelets constitutes primary hemostasis.

Secondary hemostasis stabilizes the platelet mass by forming a fibrin clot. The fibrin clot is the end product of a series of reactions involving plasma proteins. The process is known as blood coagulation. Among the plasma proteins involved are the activated forms of the clotting factors II, VII, IX, X, XI, and XII (the activated forms have an “a” following the Roman numeral, e.g., factor IIa). The activated forms of these proteins are serine proteases.

Fibrin is formed from fibrinogen, a large circulating plasma protein, by specific proteolysis. In the process, the protein thrombin (factor IIa) is consumed.

Fibrin monomers next spontaneously associate to form polymers and form a loose reinforcement of the platelet plug. Fibrin polymers are then cross-linked by certain enzymes. The fibrin polymer also traps red cells and white cells to form a finished clot.

Under normal conditions of hemostasis, an individual experiencing bleeding benefits from the ability of platelets to change shape, adhere, spread, release chemical messengers and activators, aggregate, and assemble with fibrin. This series of events stops bleeding at the site of injury and initiates the process of wound healing.

But platelet activation and clot formation can also place a person at risk of pathological cardiovascular events. For example, venous blood clot formation in the legs, a condition known as deep vein thrombosis, creates the risk that the blood clots could embolize (break apart) and result in clot entrapment in the lungs or the brain, causing pulmonary embolisms and stroke-related conditions. Platelet activation and fibrin formation in other locations in some persons create aggregates and small clots in the arterial circulation that can also lead to embolization and strokes.

In addition to age and genetic and lifestyle risk factors, implanted medical devices in the blood stream also place patients at greater risk of clot formation and embolization. Each year, approximately 500,000 heart valves are implanted in the United States. Although biomaterial advancement has somewhat reduced the risk of thrombosis (clot formation), all patients with mechanical heart valves are at increased risk of clot formation, embolization, and stroke.

Arterial stents are another type of device placed in the circulatory system that place patients at risk from platelet activation. Arterial stents are placed in clogged coronary and carotid arteries to provide oxygen to cardiac tissue. They are typically around 5 mm in diameter and are made from stainless steel or other materials. Due to the introduction of a foreign material in the blood stream, platelets can become activated and attach to the wall of the stented vessel. This leads to reocclusion (restenosis) of the stented vessel, which is a very significant risk in patients with arterial stents. Restenosis in the first 28 days is reported in 0.5 to 8% of persons receiving stents. In an effort to reduce the risk of embolization and restenosis, patients receiving heart valves or arterial stents are commonly placed on anti-coagulant or platelet-inhibiting medications before, during, and after the procedures.

Current platelet inhibiting drugs fall into three groups: (1) aspirin-related drugs, which inhibit the platelet cyclooxygenase enzyme, thus reducing production of thromboxane A₂, which is a platelet activator; (2) ADP-receptor inhibiting drugs, which block a surface membrane receptor on the platelets that is involved in the activation process; (3) monoclonal antibodies that block GPIIbIIIa receptors on the platelet surface. The GPIIbIIIa receptor binds the plasma coagulation factor fibrinogen, which is involved in both aggregation and in forming a fibrin clot. All three approaches are effective in reducing platelet activation; however no intervention is successful on all patients. Aspirin is the least expensive. But the appropriate dose varies unpredictably from person to person, and up to 30% of individuals on long-term aspirin therapy do not achieve inhibition of platelet adhesion. The ADP-inhibiting drugs are more expensive than aspirin, but are gaining popularity. However, as with aspirin, the required dose and duration of therapy varies, and a large variation in platelet adhesion characteristics in patients on the drugs exists. The GPIIbIIIa-inhibiting drugs are argued to provide the greatest platelet inhibition, but they are very expensive and still suffer from patient-to-patient variability in dosing and effectiveness. Other medications are likely to emerge, but all will probably still have the patient-to-patient variability seen with other approaches.

The failure to determine the proper dose and medication to inhibit platelets can have a great cost in money, and can cause unnecessary morbidity and death. For example, patients on anti-GPIIbIIIa drugs have been reported to have from a 5.8 to 11.2% incidence of adverse reactions in the first 28 days after stenting. The adverse reactions were defined as death, myocardial infarction, or urgent need for reintervention with angioplasty procedures. The risk was even higher when patients were not treated with the drugs. (N. Engl. J. Med., 330:956-961, 1994; N. Engl. J. Med., 336:1689-96A, 1997; Lancet, 349:1429-35, 1997.) Thus, anti-platelet drugs have a large patient-to-patient variability and many patients are refractory to some anti-platelet drugs. A method is needed to monitor platelet function so the proper dose of an anti-platelet drug for a particular patient can be determined, and so a physician can determine whether a particular patient is refractory to one anti-platelet drug but responsive to another.

Devices and methods for monitoring platelet function are described in WO 2004/024026 A2. This published PCT application was extended into the United States as application Ser. No. 11/077,191, filed Mar. 10, 2005, the contents of which are hereby incorporated by reference herein.

No reliable point-of-care method currently exists to specifically determine if platelet adhesion and aggregation have been inhibited. Thus, there is a need for a method and a device to measure platelet function, and preferably to measure platelet adhesion and aggregation as part of the measurement of platelet function. The need to measure platelet function is particularly acute in patients receiving arterial stents or other cardiovascular devices, and in other persons at risk of adverse cardiovascular events. Such a method would allow an attending physician to ensure that platelet function has in fact been inhibited in a patient at risk, and to adjust pharmacologic parameters prior to implanting a cardiovascular device, which will reduce the risk of adverse events associated with platelet initiation of clot formation.

Another need to monitor platelet function arises in platelet transfusions. Platelets are harvested and used in platelet transfusions to support patients at risk of bleeding. However, platelet storage poses problems not found with the storage of whole blood or other components. Whole blood, red and white cells may be stored at 4C for weeks. However, platelets will aggregate in cold storage and when allowed to settle. Therefore, the standard means of storing platelets is at room temperature with gentle agitation. Even under these conditions, platelets lose function by about 5 days. Thus, methods and devices for monitoring platelet function are also needed to determine whether stored platelets have adequate activity to be transfused into patients.

Another need to monitor platelet function exists for patients undergoing a medical or dental procedure to evaluate their risk of excessive bleeding during the procedure.

A further need to monitor platelet function exists for patients taking aspirin in order to reduce their risk of heart attack or stroke. There are already studies showing that for some people aspirin is not effective and that these people have a higher risk of death, stroke, or heart attack than those for whom aspirin does reduce platelet reactivity. See, Gum, et al., “A Prospective, Blinded Determination of the Natural History of Aspirin Resistance Among Stable Patients With Cardiovascular Disease,” J. Am. Coll. Cardiol., 41(6):961-5 (2003).

Accordingly, a need exists for a method to measure platelet function. Preferably, the method would monitor platelet adhesion and aggregation. Preferably, the method would monitor platelet function specifically, separately from the other aspects of clotting such as blood coagulation. Preferably, the method would be inexpensive. Preferably, the method would not depend upon platelet activation by any particular chemical platelet activator or group of chemical platelet activators. Preferably, the method could be used on whole, unprocessed blood, and could produce results quickly (e.g., be used at the bedside, during a physician visit, or during a medical procedure to provide a result almost immediately). Devices used to monitor platelet function are also needed.

### SUMMARY OF THE INVENTION

The invention provides a method of monitoring platelet function comprising: passing blood removed from a mammal through a passageway comprising a spring to contact the spring to generate a platelet mass in the passageway; and monitoring the flow or composition of the blood in the passageway to detect formation of the platelet mass.

The invention provides a method of monitoring platelet function comprising: passing blood removed from a mammal through two or more passageways, the two or more passageways comprising an obstruction or irregularity, to contact the obstruction or the wall of the passageways at the irregularity, to generate a platelet mass in the two or more passageways; and monitoring the flow or composition of the blood in the passageways to detect formation of the platelet masses.

The invention also provides a device for monitoring platelet function, comprising: a fluid-tight material forming a passageway; a pump functionally linked to the passageway for pumping blood through the passageway; a spring within the passageway, arranged such that when blood is pumped through the passageway to contact the spring, a platelet mass forms on or near the spring; and a detector for detecting the flow of blood through the passageway to detect formation of the platelet mass.

The invention provides a device for monitoring platelet function, comprising: a fluid-tight material forming a passageway; a pump functionally linked to the passageway for pumping blood through the passageway; a spring within the passageway, arranged such that when blood is pumped through the passageway to contact the spring, a platelet mass forms on or near the spring; and a detector for detecting the composition of blood in the passageway to detect formation of the platelet mass.

The invention provides a device for monitoring platelet function, comprising: a fluid-tight material forming two or more passageways; two or more pumps functionally linked to the passageways for pumping blood through the passageways; wherein the two or more passageways comprise an obstruction or irregularity arranged such that when blood is pumped through the passageways to contact the obstruction or wall of the passageways at the irregularity, a platelet mass forms on the wall of the passageways at or near the irregularity or at or near the obstruction; and a detector for detecting the flow of blood through the passageways to detect formation of the platelet masses.

The invention provides a device for monitoring platelet function, comprising: a fluid-tight material forming two or more passageways; two or more pumps functionally linked to the passageways for pumping blood through the passageways; wherein the two or more passageways comprise an obstruction or irregularity arranged such that when blood is pumped through the passageways to contact the obstruction or wall of the passageways at the irregularity, a platelet mass forms on the wall of the passageways at or near the irregularity or at or near the obstruction; and a detector for detecting the composition of blood in the passageways to detect formation of the platelet masses.

The invention provides an article for use in a device for monitoring platelet function, the article comprising: a fluid-tight material forming a passageway; and a spring in the passageway, arranged such that when blood is pumped through the passageway to contact the spring, a platelet mass forms on or near the spring.

The invention provides an article for use in a device for monitoring platelet function, comprising: a fluid-tight material forming two or more passageways; wherein two or more passageways comprise an obstruction or irregularity arranged such that when blood is pumped through the two or more passageways to contact the obstruction or the wall of the passageway at the irregularity, a platelet mass forms at or near the obstruction or on the wall of the passageway at or near the irregularity.

Additional features and advantages of the invention are set forth in the description which follows and in part will be apparent from the description. The objectives and other advantages of the invention will be realized and attained by the method and device for monitoring platelet function as particularly pointed out in the written description and claims.

It is to be understood that both the foregoing general description and the following detailed description are exemplary and explanatory and are intended to provide further explanation of the invention as claimed.

### BRIEF DESCRIPTION OF THE DRAWINGS

FIGS. 1A to 1G show passageways for the passage of blood, with various types of obstructions and irregularities in the passageways.

FIG. 2 is a perspective view of a device of the invention.

FIG. 3 is a top view of the device of FIG. 1.

FIG. 4 shows a detail of the device of FIG. 1.

FIG. 5 shows a top view of the device of FIG. 1 attached to other devices shown in schematic.

FIGS. 6 and 7 show perspective views of a device of the invention.

FIGS. 8 and 9 present data relating to the invention.

FIG. 10 is a perspective view of another device of the invention.

FIG. 11 is a top view of the device of FIG. 10.

FIG. 12 shows a detail of the device of FIG. 10.

FIG. 13 shows a top view of the device of FIG. 10 attached to other devices shown in schematic.

### DETAILED DESCRIPTION OF THE INVENTION

The invention provides a method of monitoring platelet function comprising: passing blood removed from a mammal through a passageway comprising a spring to contact the spring to generate a platelet mass in the passageway; and monitoring the flow or composition of the blood in the passageway to detect formation of the platelet mass. In one embodiment, the amount of time to form a platelet mass is measured. The spring can be made of beryllium copper, gold-plated beryllium copper, or stainless steel. In one embodiment, the spring is made of passivated stainless steel. In another embodiment, the spring is mounted transversely to the passageway.

In one embodiment, the flow of the blood in the passageway is monitored. The flow of blood can be monitored by monitoring the pressure of the blood in the passageway. The pressure can be monitored with a pressure transducer. The flow can be monitored optically, e.g., with a light-emitting diode and a light detector. In one embodiment, the composition of the blood in the passageway is monitored.

In one embodiment, the passageway and blood do not comprise an added anti-coagulant. In another embodiment, the passageway does not comprise an added biological agent that activates platelets. In another embodiment, the blood does not comprise an added biological agent that activates platelets. In one embodiment, the passageway and blood do not comprise an added chemical agent that activates platelets. In an embodiment, no biological or chemical agents are added to the removed blood.

In one embodiment, less than 0.4 ml of blood is removed from the body of the mammal. In another embodiment, less than 20 μl of blood passes through the passageway. In an embodiment, the blood passes bidirectionally through the passageway.

In one embodiment, the blood is whole blood. In another embodiment, the blood is fractionated blood. In one embodiment, the mammal is treated with an anti-platelet agent. The anti-platelet agent can be a cyclooxygenase inhibitor, an ADP inhibitor, a GPIIbIIIa inhibitor, or a combination thereof. In one embodiment, the platelet mass that forms is substantially depleted in fibrin in comparison to a normal clot.

The invention provides a method of monitoring platelet function comprising: passing blood removed from a mammal through two or more passageways, the two or more passageways comprising an obstruction or irregularity, to contact the obstruction or the wall of the passageways at the irregularity, to generate a platelet mass in the two or more passageways; and monitoring the flow or composition of the blood in the passageways to detect formation of the platelet masses. In one embodiment, the amount of time to form a platelet mass is measured. In one embodiment, the formation of the platelet masses in the two or more passageways are detected simultaneously.

In one embodiment, the two or more passageways comprise an obstruction selected from a spring, wire form, metal screen, woven cloth, sheet metal with an orifice, sheet metal forms, polymeric fibers, natural fibers, cellulose fibers, metal wires, suture strands, laser etched or molded plastic formations, glass formations or glass beads. In one embodiment, the obstruction is a spring. In one embodiment, the spring is made of beryllium copper, gold-plated beryllium copper, or stainless steel. In another embodiment, the spring is made of passivated stainless steel. In another embodiment, the spring is mounted transversely to the passageway.

In one embodiment, the flow of the blood in the two or more passageways is monitored. In one embodiment, the flow is monitored by monitoring the pressure of the blood in the two or more passageways. In one embodiment, the pressure is monitored with a pressure transducer. In one embodiment, the flow is monitored optically. In one embodiment, the flow is monitored with a light-emitting diode and a light detector.

In one embodiment, the composition of the blood in the two or more passageways is monitored. In another embodiment, the passageways and blood do not comprise an added anti-coagulant. In one embodiment, the passageways do not comprise an added biological agent that activates platelets. In another embodiment, the blood does not comprise an added biological agent that activates platelets. In one embodiment, the passageways and blood do not comprise an added chemical agent that activates platelets. In another embodiment, no biological or chemical agents are added to the removed blood.

In one embodiment, less than 0.4 ml of blood is removed from the body of the mammal. In another embodiment, less than 50 μl of blood passes through the two or more passageways. In one embodiment, at least 15 μl of blood passes through each passageway. In an embodiment, the blood passes bidirectionally through the two or more passageways.

In one embodiment, the blood is whole blood. In another embodiment, the blood is fractionated blood.

In one embodiment, the mammal is treated with an anti-platelet agent. In another embodiment, the anti-platelet agent comprises a cyclooxygenase inhibitor, an ADP inhibitor, a GPIIbIIIa inhibitor, or a combination thereof. In one embodiment, the platelet masses that form are substantially depleted in fibrin in comparison to a normal clot.

The invention provides a device for monitoring platelet function, comprising: a fluid-tight material forming a passageway; a pump functionally linked to the passageway for pumping blood through the passageway; a spring within the passageway, arranged such that when blood is pumped through the passageway to contact the spring, a platelet mass forms on or near the spring; and a detector for detecting the flow of blood through the passageway to detect formation of the platelet mass.

The invention provides a device for monitoring platelet function, comprising: a fluid-tight material forming two or more passageways; two or more pumps functionally linked to the passageways for pumping blood through the passageways; wherein the two or more passageways comprise an obstruction or irregularity arranged such that when blood is pumped through the passageways to contact the obstruction or wall of the passageways at the irregularity, a platelet mass forms on the wall of the passageways at or near the irregularity or at or near the obstruction; and a detector for detecting the flow of blood through the passageways to detect formation of the platelet masses.

The invention provides a device for monitoring platelet function, comprising: a fluid-tight material forming two or more passageways; two or more pumps functionally linked to the passageways for pumping blood through the passageways; wherein the two or more passageways comprise an obstruction or irregularity arranged such that when blood is pumped through the passageways to contact the obstruction or wall of the passageways at the irregularity, a platelet mass forms on the wall of the passageways at or near the irregularity or at or near the obstruction; and a detector for detecting the composition of blood in the passageways to detect formation of the platelet masses.

The invention provides an article for use in a device for monitoring platelet function, the article comprising: a fluid-tight material forming a passageway; and a spring in the passageway, arranged such that when blood is pumped through the passageway to contact the spring, a platelet mass forms on or near the spring. The spring can be made of beryllium copper, gold-plated beryllium copper, or stainless steel. The spring can be made of passivated stainless steel. The spring can be mounted transversely to the passageway. The length of the spring can be from 0.025 cm to 0.15 cm.

The invention provides an article for use in a device for monitoring platelet function, comprising: a fluid-tight material forming two or more passageways; wherein two or more passageways comprise an obstruction or irregularity arranged such that when blood is pumped through the two or more passageways to contact the obstruction or the wall of the passageway at the irregularity, a platelet mass forms at or near the obstruction or on the wall of the passageway at or near the irregularity. In one embodiment, the two or more passageways comprise an obstruction selected from a spring, wire form, metal screen, woven cloth, sheet metal with an orifice, sheet metal forms, polymeric fibers, natural fibers, cellulose fibers, metal wires, suture strands, laser etched or molded plastic formations, glass formations or glass beads. In one embodiment, the obstruction is a spring. The spring can be made of beryllium copper, gold-plated beryllium copper, or stainless steel. In one embodiment, the spring is made of passivated stainless steel. In another embodiment, the spring is mounted transversely to the passageway. In one embodiment, the length of the spring is from 0.025 cm to 0.15 cm.

The invention provides methods and devices for assessing platelet function, as evidenced by platelet adhesion, and preferably platelet aggregation. In the methods, blood is drawn through a passageway, such as a catheter, past or against an obstruction or irregularity in the passageway, such as a wire placed in the catheter. The platelets adhere and aggregate on the obstruction or on the wall of the passageway near the obstruction or irregularity, and form a platelet mass. It is believed that shear forces associated with passing or contacting the obstruction or irregularity in the passageway activate the platelets and induce them to adhere to the foreign material of the obstruction or the walls of passageway and to aggregate. When the platelet mass forms, it occludes the lumen of the passageway and flow stops or slows. The time of partial or full occlusion of the lumen can be recorded as the platelet mass formation time.

Since a platelet mass is the end product of platelet activity, formation of a platelet mass depends on the functioning of all platelet activities, including platelet adhesion and, if the platelet mass is thicker than about 15 microns, platelet aggregation. (If the platelet mass is thicker than about 15 microns, it involves more than a layer of platelets that forms due to platelet adhesion to a surface, but rather involves a mass formed by platelet-to-platelet aggregation.) This contrasts with some current platelet tests that measure only one specific platelet activity, such as release of a particular biochemical, or depend only on platelet adhesion and not aggregation. It has been found that the platelet mass in the methods of the present invention contains little or no fibrin or red or white blood cells. Thus, in at least some embodiments, the methods of the invention measure platelet function specifically, independently of the blood coagulation reactions.

No chemical or biological platelet activators need to be added to the blood or the passageway for the present methods, although in some embodiments they optionally can be added. Thus, the methods do not depend on platelets responding to a particular biochemical activator or particular group of activators. The methods are fast and can use whole unprocessed blood. Accordingly, they can produce results quickly and inexpensively with a small sample of blood taken at the patient's bedside, during a physician visit, or during an interventional procedure.

Definitions: “Platelet function” refers to platelets adhering to a substrate, changing shape, releasing chemical messengers or clotting factors stored in the cytoplasm of the platelets, and/or aggregating with other platelets. “A biological or chemical agent that activates platelets” refers to a substance that upon contact with platelets induces platelets to perform any of those platelet functions (without a requirement that the platelets be exposed to shear or any other mechanical activator).

The term “a biological agent that activates platelets” refers to an agent found naturally in a mammalian body that has the biological role of activating platelets, such as collagen, ADP, thrombin, thromboxane A₂, serotonin, and epinephrine.

“A chemical agent that activates platelets” refers to a compound that activates platelets other than a mammalian biological agent. It includes, e.g., non-biological synthetic compounds, derivatives of biological agents that activate platelets, or biological agents found in plants or microorganisms that activate platelets.

“An added biological or chemical agent” refers to a compound or substance that is added to the blood after removal from the body. An “added agent in the passageway” refers to an agent placed or incorporated in the passageway prior to addition of blood to the passageway. The agent could be, for instance, adhered to the wall of the passageway or to an obstruction in the passageway.

“Obstruction” refers to an object that partially or fully obstructs the passageway. Preferably the obstruction partially obstructs the passageway. Examples of obstructions include (a) a plug, such as a wire, that occupies a portion of the passageway (preferably with a space between the plug and the wall of the passageway), (b) a filter or screen, (c) a fiber, or (d) a spring.

As used herein, an obstruction in the passageway that is a “plug” is a solid nonporous object that partially or fully obstructs the passageway. The plug can be any shape in cross-section, e.g., circular, square, or rectangular, and can be composed of any non-porous material, e.g., plastic or metal.

“Blood” as used herein refers to whole blood or to a blood fraction containing platelets. Preferably, blood is removed from the mammal and then passed through the passageway in the methods of the invention without any processing and without the addition of any agents (e.g., anti-coagulants or platelet activators). However, the method will also work with purified platelets or with any blood fraction enriched in platelets or containing platelets. Accordingly, the term “blood” includes platelet-containing plasma, purified platelets, or any blood fraction containing platelets. The term “whole blood” refers to blood that has not been fractionated.

A “platelet mass” as used herein refers to any mass that is predominantly platelets. The mass can also contain fibrin and other cells. Preferably, it is depleted in fibrin and depleted in other cells as compared to a natural clot. A platelet mass can be less than about 15 microns thick in one or more dimensions, i.e., consisting of a layer of platelets about 5 or fewer platelets thick and formed by platelet adhesion, with little or no platelet-to-platelet aggregation. Preferably, however, the platelet mass is thicker than about 15 microns in all dimensions. The term “platelet plug” is used interchangeably with “platelet mass.”

The invention provides a method of monitoring platelet function in a mammal involving passing blood removed from the body of the mammal through a passageway to contact an obstruction or irregularity in the passageway to generate a platelet mass in the passageway, and monitoring the flow or composition of blood in the passageway. The formation of a platelet mass causes a change in the flow or composition of the blood in the passageway, and the change in flow or composition is detected.

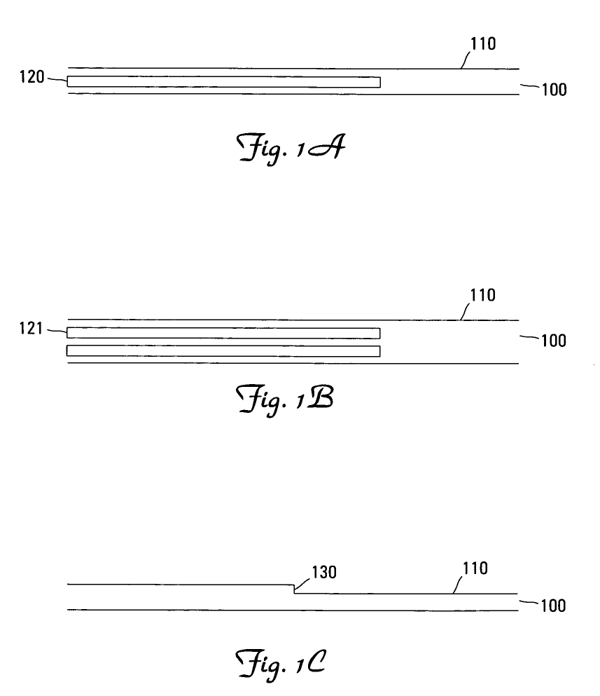

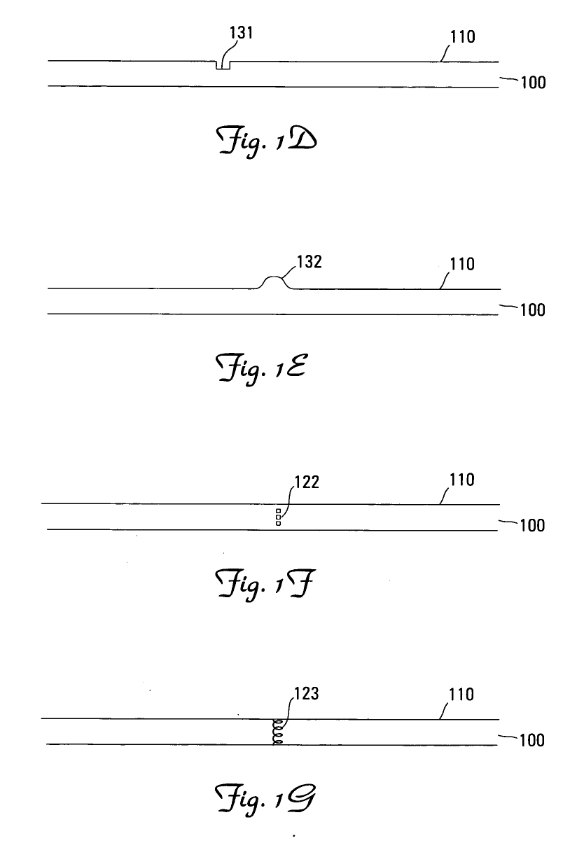

In devices of the invention, blood passes through a passageway 100, formed by fluid-tight walls 110 of a foreign material (i.e., any material other than the endothelium of a natural blood vessel). See FIG. 1A. Preferably, the foreign material is a non-biological material. It can be, for instance, any type of plastic, glass, rubber, TEFLON, or metal. Within the passageway is an obstruction or irregularity. A passageway with obstruction 120 is shown in FIG. 1A. The obstruction is also preferably made of a foreign material. It can be porous or non-porous. It can be the same material as the wall of the passageway or a different material.

Blood is pumped through the passageway to contact the obstruction or the wall of the passageway at the irregularity. The obstruction or irregularity creates areas of high shear and low shear for fluids passing through the passageway. It is believed that high shear activates the platelets and areas of low shear allow the platelets to adhere and form a platelet mass. Preferably, the blood is pumped past the obstruction or irregularity, until a platelet mass forms that prevents or resists blood passing.

However, the obstruction can totally occlude the passageway, and the irregularity can be a closed end of the passageway, where blood can not pass the obstruction or irregularity. In that case, blood can be passed back and forth against the occluding obstruction or irregularity until a platelet mass forms that is detected.

One example of an obstruction is a wire 120 as shown in FIG. 1A. The obstruction preferably only partially obstructs the passageway. Preferably the obstruction leaves a gap of at least about 20 microns between the obstruction and the passageway wall. Thus, in that case, in order to fully occlude the passageway the platelet mass must be at least about 20 microns thick. To form a mass that size, the platelets must not merely adhere to the surface but must also aggregate to each other. Thus, the method tests the ability of the platelets to show both the activity of adhering and the activity of aggregating.

As blood is pumped past the obstruction 120, a platelet mass is formed on or near the obstruction. Typically, the platelet mass forms at a location of low shear, such as on the end of a wire obstruction. Platelet function can be monitored by measuring the time until partial or full occlusion of the passageway. Occlusion of the passageway can be detected by any suitable means. For instance, a light-emitting diode and a coupled detector can be placed across one point of the passageway to detect passing of the red blood past that point. A pressure transducer can be used to monitor the pressure needed to pump the blood. The passageway can be placed across the light path of a spectrophotometer, so that the spectrophotometer detects (a) the passing of red blood past the light path, (b) an increase in scattering and/or a change in color at the point of the platelet plug as the platelet plug develops, if the light path is positioned to pass through the expected point where the platelet plug forms, or (c) a change in color of the blood outside of the platelet plug associated with the formation of the platelet plug. The time it takes the blood to pass from point A to point B can be measured. Chemical sensors can also be used to measure the concentrations of particular biochemicals that change, either in the blood as a whole or in microenvironments at or near the platelet mass, as the platelet mass forms. For instance, pH, Mg⁺⁺ concentration, K⁺ concentration, Na⁺ concentration, O₂ concentration, or CO₂ concentration can be monitored by sensors and methods known in the art.

The dimensions of the passageway and obstruction or irregularity can be any dimensions suitable, i.e., wide enough to allow blood to pass freely through the passageway until a platelet mass forms, and narrow enough that upon formation of a platelet mass the occlusion of the passageway can be detected. For instance, the passageway can be a millimeter or less in diameter or more than a cm in diameter. A wire obstruction of the passageway can leave, for instance, a gap of about 50 microns with the passageway wall. Other larger and smaller gap sizes and dimensions are also possible.

Blood can be pumped bidirectionally or unidirectionally through the passageway. Pumping the blood bidirectionally, i.e., back and forth past the obstruction or irregularity, has the advantage that it allows a smaller volume of blood to be used. Also, with bidirectional flow, any platelet mass formation time can be measured with a finite amount of blood. With unidirectional flow of blood through a linear passageway that is open at both ends, longer platelet mass formation times will require the use of more blood. Pumping blood unidirectionally through a closed loop, where the blood can cycle the loop as many times as necessary, has the same advantages as bidirectional flow, namely allowing the use of smaller volumes of blood and allowing measurement of extended plug formation times.

Thus, some embodiments of the devices and methods of the invention allow the use of small volumes of blood to monitor platelet activity. Specifically, in some embodiments, less than about 2 ml, less than about 1 ml, less than about 0.4 ml, less than about 0.2 ml, less than about 0.1 ml, or less than 50 μl is used. In some embodiments 10 to 40 μl is used. In some embodiments, 20 μl is used. In some embodiments, a drop, such as is formed by a finger prick, can be used.

Certain embodiments of the obstruction or irregularity are shown in FIGS. 1A to F. FIG. 1A shows a wire 120 as an obstruction. The wire 120 can be centered or off-center in the passageway. Either or both of the passageway 100 and wire 120 can have non-circular cross-sections. The wire 120 in this embodiment can be replaced with a plug of any non-porous material. The wire can be any length, and can be shorter than it is wide. The obstruction can be multiple wires or plugs 121, as shown in FIG. 1B.

The passageway can comprise an irregularity rather than, or in addition to, an obstruction. The irregularity can be any angle, narrowing, expansion, or curve in the passageway that is suitable to allow formation of a platelet mass. For instance, the irregularity can be step 130 in the wall of the passageway, as shown in FIG. 1C. The smaller diameter section of the passageway could be on the same center as the larger diameter section, or offset. The irregularity could be a narrowed section 131 of the passageway, as shown in FIG. 1D. The irregularity could also be an expansion 132 in the passageway 100 (FIG. 1E).

Another example of a suitable obstruction is an inserted flow restrictor 122 (FIG. 1F). The flow restrictor could be, for example, a filter membrane; a single filter or a plurality of fibers, wires, or ribbons; or a piece of woven or knitted fabric.

Another example of a suitable obstruction is spring 123 (FIG. 1G). The spring can be made of any suitable metal such as beryllium copper, gold-plated beryllium copper, stainless steel, etc.

A plurality of obstructions or irregularities, or a combination of both obstructions and irregularities can be used. The passageway in the invention can be circular, square, or any other shape in cross-section. The passageway can be curved or linear. Any flow pattern can be used that produces a platelet mass in a suitable time.

For instance, steady unidirectional, or oscillating bidirectional flow can be used. With oscillating bidirectional flow, the oscillation pattern can be sinusoidal, saw tooth, square wave, asymmetric saw tooth, trapezoidal, asymmetric trapezoidal, or other patterns. In unidirectional flow, a pulsate component can be superimposed on the steady flow, and the pulsate component can have any of the above patterns. The flow patterns can also vary with time or with measured resistance to reduce the risk of dislodging a platelet mass once it has started to form. Dwell periods (no flow) can be introduced to allow aggregation of platelets activated by shear. To achieve the flow patterns described, a pump is preferably used to draw a predetermined volume of blood at a predetermined flow rate (although the flow rate can vary with time, as described above) and a predetermined shear rate into and through the passageway.

One embodiment of an article for use in a device for monitoring platelet function is composed of a rigid precision-molded plastic piece, with a passageway molded therein. The article can have an aperture for accepting blood, linked to the passageway. The ends of the passageway can be open to the air to allow free flow of blood without pressure buildup. The passageway in one embodiment is about a millimeter in diameter and a few cm long, with a stainless steel wire plug of a few millimeters length fixed to one wall of the passageway. The gap between the wire plug and the other wall of the passageway can be, for example, about 50 microns.

The article can be placed in a flow detection device, where the device includes a bidirectional pump linked to the passageway and an LED and a coupled detector are placed across one end of the passageway. The detector detects the passing of blood and then air, as the blood is pumped back and forth, until a platelet mass forms and prevents the passing of blood. The article can be made of inexpensive plastic so it is disposable.

One of the advantages of the invention is that no biological or chemical agent that activates platelets must be added to the blood or to the passageway through which blood is pumped. Thus, in some embodiments of the invention, the passageway (prior to addition of blood) does not contain an added biological agent that activates platelets. The blood also optionally does not contain an added biological agent that activates platelets. In some embodiments both the passageway and blood do not have an added biological agent that activates platelets. In some embodiments, either or both of the passageway and blood do not comprise an added chemical agent that activates platelets. In some embodiments, the passageway does not comprise a biological component to which platelets naturally adhere. In specific embodiments, the passageway does not comprise collagen, ADP, epinephrine, or a derivative thereof. In some embodiments, no biological or chemical agents are added to the removed blood. For instance, in some embodiments, no anti-coagulants are added to the removed blood. In some embodiments, the passageway and blood do not comprise an added anti-coagulant.

However, the methods optionally can also involve use of an added agent that activates platelets. The agent can be added to the blood after it is removed from the body of the mammal, or it can be added to the passageway of the device and thus added to the blood as the blood passes through the passageway. For instance, the walls of the passageway, or the walls of an obstruction can be coated with the agent.

If the obstruction is a filter, the filter could be soaked in the agent. Among the agents that could be used are thromboxane A₂. Aspirin is believed to inhibit platelet function primarily by inhibiting production of thromboxane A₂, so in some embodiments of testing the effectiveness of aspirin therapy, it may be useful to add thromboxane A₂ to the blood or passageway. In particular, it may be useful to compare the platelet mass formation time with and without thromboxane A₂ added to the blood or passageway.

Other agents that can be added to the removed blood or to the passageway in some embodiments include any of the activators of platelets. Among these are ADP, collagen, thrombin, epinephrine, and serotonin. Other compounds that are not platelet activators but are beneficial to plug formation could also be added. These include fibrinogen, fibrin, and von Willebrand factor.

The invention can be used to monitor platelet function of patients treated with ADP inhibitors. Among these drugs are clopidogrel (PLAVIX) and ticlopidine. In the case of patients treated with ADP inhibitors, if a platelet-activating agent is added to the removed blood or the passageway, ADP may be useful as the added agent. In particular, it may be useful to compare the platelet mass formation time with and without ADP added to the blood or passageway.

The invention can also be used to monitor platelet function of patients treated with GPIIbIIIa inhibitors. Among the GPIIbIIIa inhibitors are tirofiban, eptifibatide, and abciximab. In the case of patients treated with GPIIbIIIa inhibitors, if a platelet-activating agent is added to the removed blood or the passageway, fibrinogen may be a preferred agent since it binds to the GPIIbIIIa receptors.

Platelet function can be monitored using a method comprising: (a) passing blood removed from a mammal through a passageway comprising an obstruction or irregularity to contact the obstruction or the wall of the passageway at the irregularity, to generate a platelet mass in the passageway; and monitoring the flow or composition of the blood in the passageway to determine a platelet mass formation time, wherein the blood and passageway do not comprise an added biological or chemical agent that activates platelets; and (b) passing blood removed from a mammal through a passageway comprising an obstruction or irregularity to contact the obstruction or the wall of the passageway at the irregularity, to generate a platelet mass in the passageway; and monitoring the flow or composition of the blood in the passageway to determine a platelet mass formation time, wherein the blood and passageway comprise an added biological or chemical agent that activates platelets; and (c) comparing the platelet mass formation times. The biological or chemical agent that activates platelets can be, for instance, thromboxane A₂, ADP, or fibrinogen.

It has been found that the platelet mass formed in some embodiments of the invention is substantially free of fibrin and of red and white blood cells. Thus, in some embodiments, the platelet mass is substantially depleted in fibrin in comparison to a natural clot. For instance, the platelet mass can contain less than about 50%, less than about 30%, less than about 10%, or less than about 5% of the fibrin per unit mass found in a natural clot in the peripheral blood system. In other embodiments, the platelet mass has no detectable fibrin. In certain embodiments, the platelet mass is substantially depleted in red cells and/or white cells (e.g., contains less than about 50%, less than about 30%, less than about 10%, or less than about 5% of the red or white cell found in a natural clot in the peripheral blood stream or has no detectable red or white cells).

In some embodiments of the invention the blood passes (e.g., is pumped past) the obstruction or irregularity in the passageway.

Platelet mass formation can be detected by monitoring the flow or the composition of the blood in the passageway. In some embodiments, the flow is monitored. Flow can be monitored, for instance, by monitoring the pressure of the blood in the passageway or optically. The pressure can be monitored with a pressure transducer. Optical monitoring can be, for instance, with a LED and a coupled light detector. The optical monitoring, or other methods, can be used to measure the time for blood to travel a certain distance in the passageway. Flow can also be monitored by a flow meter or by volume displacement, as well as by other means known to those of skill in the art.

In some embodiments, the composition of the blood in the passageway is monitored. For instance, formation or size of the platelet mass can be directly monitored, e.g., by optical means such as with an LED or a spectrophotometer. The chemical composition of the blood can also be monitored. For instance, pH or concentration of O₂, CO₂, Mg⁺⁺, or K⁺ can be monitored, as these correlate with platelet mass formation.

In some embodiments, the passageway comprises an obstruction. The obstruction can be, for instance, a plug. The plug can be a metal wire, plastic, ceramic, glass, or any non-porous substance. The plug can fully or partially obstruct the passageway.

In some embodiments the platelet mass develops thickness in all dimensions. That is, these embodiments of the methods require platelet aggregation in addition to platelet adhesion. Thus, in some embodiments, the platelet mass has a thickness in all dimensions of at least about 20 microns, at least about 30 microns, at least about 40 microns, at least about 50 microns, at least about 70 microns, or at least about 100 microns.

In some embodiments of the invention, the passageway does not comprise a biological component to which platelets naturally adhere. In some embodiments, the passageway does not comprise collagen, ADP, epinephrine, or a derivative thereof. In some embodiments, the passageway and blood do not comprise an added anti-coagulant.

In some embodiments of the methods, the method further comprises adding a platelet activator to the blood. In some embodiments the passageway comprises a platelet activator. The platelet activator can be, for instance, thromboxane A₂.

In some embodiments of the methods and devices of the invention, the platelets are activated at least partially by mechanical forces. In some embodiments, the platelets are activated solely by mechanical forces. It is believed that the platelets in the methods of the invention are activated by high shear and adhere at a point of low shear. However, by varying the dimensions of the passageway, the velocity of flow generated by the blood pumping, and the material of the walls of the passageway and of any obstructions (e.g., the adhesiveness of the material), wide ranges of shear can be used. Maximum shear rates in different devices in which platelet mass formation was detected spanned at least the range of 50 to 5,000 sec⁻¹.

In some embodiments, less than 2 ml, less than 1 ml, less than 0.4 ml, less than 0.2 ml, less than 0.1 ml, or less than 50 μl is removed from the body of the mammal. In some embodiments, less than these amounts are transferred to the passageway.

In some embodiments of the invention, the blood passes bidirectionally through the passageway. In other embodiments, at least part of the passageway is a loop (i.e., a closed circuit, whether circular, oval, square, or another shape) and the blood passes unidirectionally through the loop.

In some embodiments of the invention, the blood is whole blood. In some embodiments, the removed blood is fractionated before being used in the methods and devices of the invention.

In some embodiments of the devices and articles of the invention, the device or article further comprises a fluid-tight material forming an aperture linked to the passageway.

In some embodiments of the devices of the invention, the device operates without a biological agent that activates platelets. In some embodiments, the device operates without a chemical agent that activates platelets.

In some embodiments of the devices and articles of the invention, the obstruction in the passageway is arranged such that when blood is pumped through the passageway to contact the obstruction, a platelet mass that is substantially free of fibrin and is at least about 20 micron thick in all dimensions forms on or near the obstruction.

In some embodiments of the devices and articles of the invention, the irregularity in the passageway is arranged such that when blood is pumped through the passageway to contact the wall of the passageway at the irregularity, a platelet mass that is substantially free of fibrin and that is at least about 20 micron thick in all dimensions forms on the wall of the passageway at or near the irregularity.

In some embodiments, the blood flows past the obstruction or irregularity, and the obstruction or irregularity leaves a passageway at least 20 microns in diameter or width at the obstruction or irregularity. For instance, the gap between a plug and the wall of the passageway is at least 20 microns in these embodiments. For another example, the diameter or width of the passageway at the narrowest point of the passageway at an irregularity that narrows the passageway is at least 20 microns in these embodiments. When a platelet plug forms that fills the passageway at this point, the passageway is occluded and this is detected as a change in the flow of the blood in the passageway. Thus, the method detects the formation of a platelet plug at least 20 microns thick. In other embodiments, the obstruction or irregularity leaves a passageway of at least 50 microns, at least 100 microns, 20 to 100 microns, or 20 to 200 microns in diameter or width at the obstruction or irregularity.

In some embodiments of the invention, the mammal whose platelet function is monitored is treated with an anti-platelet agent. In particular embodiments, the anti-platelet agent comprises a cyclooxygenase inhibitor (e.g., aspirin or other salicylates), an ADP inhibitor, a GPIIbIIIa inhibitor, or a combination thereof.

Several uses of the methods and devices of the invention exist. The methods and devices can be used to monitor the effectiveness of anti-platelet agents in patients treated with anti-platelet agents. Such patients include those treated by interventional cardiology catheterization. This includes angiograms, angioplasty, and stent placement. In addition, the methods can be used to monitor the effectiveness of anti-platelet agents in patients who receive an artificial heart valve.

The methods and devices can be used to monitor the effectiveness of aspirin or other anti-platelet agents in patients taking the agents to prevent a cardiovascular event, such as coronary thrombosis (heart attack), pulmonary embolism, stroke, or deep vein thrombosis due to excessive platelet activity.

The methods and devices can be used to test patients for their risk of excessive bleeding. This testing can be needed, for instance, prior to a surgical or dental procedure. For instance, the methods can be used on patients prior to having a tooth pulled or wisdom tooth removed to determine their risk of excessive bleeding. If it is determined that the patient is at risk of excessive bleeding, appropriate precautions can be taken, such as doing the procedure in a setting where a blood transfusion or platelet transfusion is available.

The methods can also be used to monitor liver function. When liver function falls, blood flow through the spleen increases. The spleen, which normally degrades old non-functional platelets, then begins to degrade good platelets as well and the platelet count falls. Since a fall in platelet function can be due to low platelet count, by detecting low platelet function the present methods provide a quick way of detecting possible low platelet count. Accordingly, they can be used to screen for liver disease including hepatitis A, B, and C, cirrhosis, and liver damage due to alcoholism.

Platelet Reactivity Test (PRT) and Cartridge

The PRT test is used to activate platelet gel formations using a small (20 microliter) whole blood sample to help identify proper platelet function in the absence of platelet drug therapy and to identify inhibited platelet function in the presence of platelet drug therapy. The test is performed by running a small quantity of blood through a channel restriction that will induce the formation of platelet gels and trap them in a focused area similar to the way platelets are activated and focused in the body.

The cartridge can be a single channel, preferably using 20 μl of blood, or dual channel cartridge, preferably using 40 μl of blood. The cartridge is injection molded from common plastic material. The design is constructed to accept a common 75 mm capillary tube which is bonded into the cartridge using a common adhesive. The main channel is approximately 0.020 inch (0.051 cm) deep by 0.035 inch (0.089 cm) wide. The main channel is used to transport a blood sample to a restriction area located within the main channel overall length. The restriction area is used to create shear stress within the blood sample which in turn will activate the platelets and form a platelet gel within the restriction. Shorter (0.030 inch (0.076 cm)) and longer (up to 0.400 inch (1.02 cm)) restriction channel lengths, as well as a wider (up to 0.013 inch (0.033 cm)) channel width and a deeper (up to 0.018 inch (0.046 cm)) restriction channel depth were used but the results were not as consistent. The currently preferred restriction channel area is 0.010 inch deep by 0.010 inch wide by 0.080 inch long (0.025 cm deep by 0.025 cm wide by 0.20 cm long).

Several different cartridge materials including polycarbonate, polyester, acrylic, and polystyrene were tried, but no significant differences were seen. The current cartridge design uses polystyrene as the base material.

As the channel restriction activates the platelets there is a need for a platelet trap to focus the formation of the platelet gel clot. The platelet trap is located within the 0.080 inch (0.20 cm) length restriction area. The trap may be oriented with its axis perpendicular or oblique to the channel axis and at an angle to the cartridge surface which is parallel, perpendicular, or in between. The trap can take the form of a spring, wire form, metal screen, woven cloth, sheet metal with an orifice, sheet metal forms, polymeric fibers, natural fibers, cellulose fibers, metal wires, suture strands, laser etched or molded plastic formations, glass formations or glass beads. Surface modification such as chemical etching, coatings, surfactant wash, and positively charging the surface may also be used as a trap.

Different fibers have been stretched across the channel, including polypropylene, silk, gut, polyester, cotton, rayon, cellulose acetate, stainless steel wire, zinc wire, beryllium copper wire, copper wire, copper nickel wire, tungsten wire, gold-plated tungsten wire, and platinum wire. Although there were small subtle differences in the effectiveness of the fiber strands, the diameter of the fiber seemed to have the most influence. Preferred fiber diameters are 0.001 to 0.003 inch (0.0025 to 0.0076 cm). The diameters that worked very well were diameters in the range of 0.0012 to 0.0018 inch (0.0030 to 0.0046 cm). The most preferred diameter is 0.0015 inch (0.0038 cm). Several design configuration were explored including: one fiber in the middle, one fiber on the channel ends, fibers which were cut to create fiber fingers in the channel, criss-crossed fibers, a fiber formed into a loop, a diagonal fiber, and one fiber sticking up from the base of the channel. Eventually it was discovered that fiber positioning within the channel restriction was very important specifically the closer the fiber was located to the center of the channel restriction the more consistent the results were. To circumvent the issues that could cause functional and manufacturing problems it was decided that the most preferred embodiment includes placing a spring across the channel with its axis either vertical or horizontal.

Several different spring materials were tried, including beryllium copper, gold-plated beryllium copper, and stainless steel. Although no particularly significant differences in performance were found, passivated stainless steel wire with a nominal wire diameter of 0.0015 inch (0.0038 cm) worked the best. Spring outside diameter, length, and pitch of the spring strongly influence the performance. The outside spring diameter of 0.010 inch (0.025 cm) must match the restriction channel depth closely. If the spring diameter is smaller than the depth of the channel by 0.001 inch (0.0025 cm), the spring is more likely to trap an air bubble.

In a preferred embodiment, the spring is staked or has a slight compression fit within the channel. In order to accomplish this, the channel was modified to include a small recess area that will hold the two end coils to prevent the spring from twisting on its axis. This meant that the length of the spring must be sufficient to provide a slight compression fit between the two ends of the spring recess area. If the spring is too short, it will allow the spring to twist in the channel and provide a shunt path in which the blood can bypass the platelet trap. The current spring length which works best is 0.016 to 0.018 inch (0.041 to 0.046 cm) to match the 0.0135 inch (0.034 cm) spring recess length. The spring pitch and number of total coils is also important. If the gaps between the spring wires get too small, the spring might not provide the proper discrimination between people on aspirin and people not on aspirin. If the spring pitch is not consistent, the blood might not flow uniformly through the spring which can also affect the performance. One spring design functions with 4.5 total coils, 2.5 active coils, and a nominal pitch of 0.0045 inch (0.012 cm) in a 0.015 inch (0.038 cm) spring recess length. The current spring design functions best with 4.0 total coils, 2.0 active coils, and a nominal pitch of 0.0053 inch (0.013 cm).

Thus, the preferred spring is made of passivated stainless steel wire with a nominal wire diameter of 0.0015 inch (0.0038 cm), an outside spring diameter of 0.0105 inch (0.027 cm), and a length of 0.016 to 0.018 inch (0.041 to 0.046 cm). This preferred spring has 4.0 total coils, 2.0 active coils, and a nominal pitch of 0.0053 inch (0.013 cm).

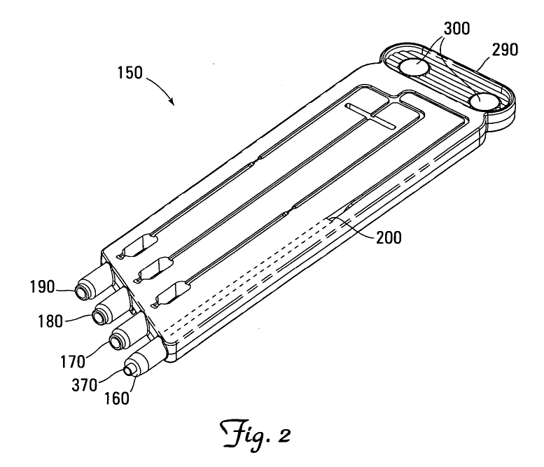

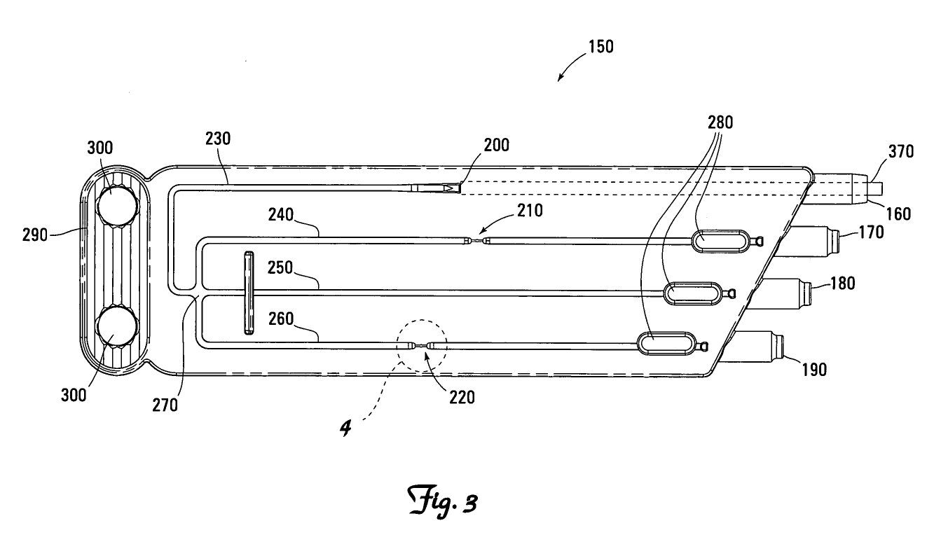

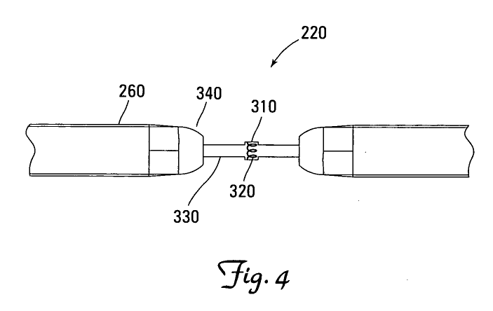

An example of a device for monitoring platelet function of the invention is shown in FIGS. 2 and 3. Cartridge 150 has capillary tube inlet 160 that can receive a capillary tube filled with blood. A capillary tube filled with blood can be attached to capillary tube port 200. Conduit 230 leads from capillary tube port 200 to junction 270 and junction 270 splits into conduit 250 and passageways 240, 260. Conduit 250 and passageways 240, 260 include overflow wells 280. Conduit 250 and passageways 240, 260 lead to ports 180, 170, 190, respectively. Passageways 240 and 260 include platelet gel formation portions 210 and 220, which include constrictions 340, narrower conduits 330, spring housing 310, and springs 320. See FIG. 4. Cartridge 150 includes tab 290 and holes 300.

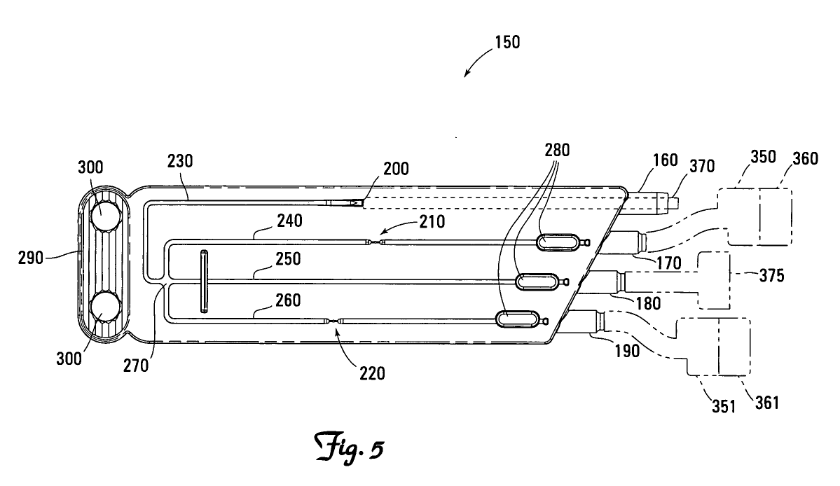

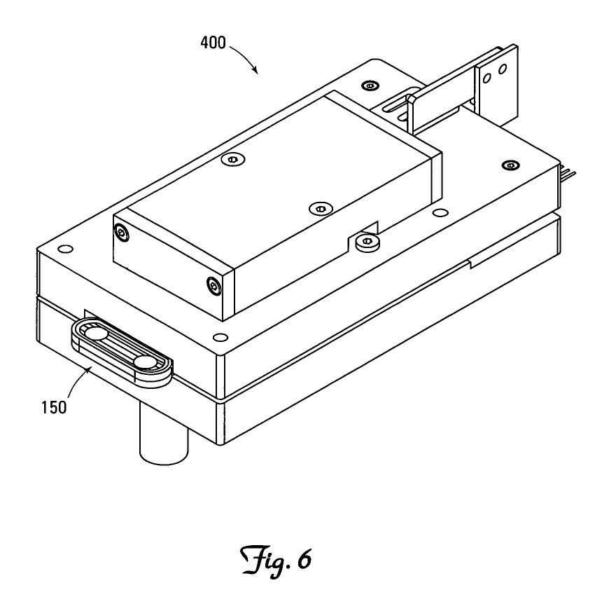

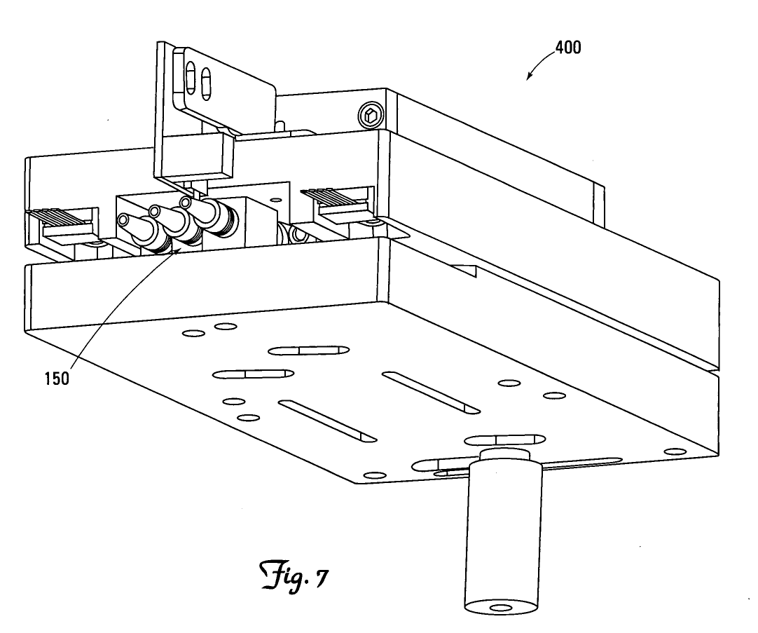

In use, the cartridge is secured in instrument 400 and preheated for three to five minutes at 33C. See FIGS. 6 and 7. The cartridge is then removed from the instrument 400 and 40 μl of blood previously taken from a subject by a pinprick is placed in capillary tube 370, which is already bonded to the capillary tube port 200. See FIG. 5. The cartridge 150 is then placed back in the instrument 400. The instrument 400 is maintained at 33C. In a preferred embodiment, the cartridge 150 has a bar code that is read by the instrument 400. The instrument 400 will reject the cartridge 150 if the cartridge has been out of the instrument for more than two minutes after the preheating step.

Bidirectional pumps 350, 351 are attached to ports 170, 190, respectively. The bidirectional pumps 350, 351 are coupled to pressure transducers 360, 361 to measure the pressure in passageways 240, 260. Pumps 350 and 351 can be diaphragm pumps driven by a stepper motor. A solenoid valve 375 is attached to port 180.

The cartridge 150 is filled with blood as follows. Solenoid valve 375 is closed. Pump 351 is off and pump 350 is started. Blood flows into passageway conduit 240 and when the blood reaches a predetermined point, the solenoid valve 375 is opened so an air bubble is pulled behind the blood in passageway 240. Pump 350 is then stopped and solenoid valve 375 is closed. Pump 351 is started and blood flows into passageway 260. When the blood reaches a predetermined point in passageway 260, the solenoid valve 375 is opened so an air bubble is pulled behind the blood in passageway 260. The solenoid valve 375 is then closed and the bidirectional pumps 350 and 351 reverses direction and pump some air back towards the capillary tube 370. Approximately 15 to 20 μl of blood is now in each of passageways 240, 260. The amount of blood in each passageway can be different.

Next, the solenoid valve 375 is opened and the bidirectional pumps 350, 351 simultaneously cycle the blood back and forth in the passageways 240 and 260 between predetermined points. A gel forms and the pumps 350, 351 operate until an end point is reached. The end point can be based on pressure, e.g., when the pressure has doubled, or on resistance to flow (pressure divided by velocity), e.g., when the resistance to flow is twice the initial resistance to flow. The time it takes to reach the end point is the platelet reactivity time.

The instrument 400 will report the average of the platelet reactivity times determined from the blood in passageways 240 and 260. Typical platelet reactivity times range from 10 to 400 seconds. If the two platelet reactivity times determined from the blood in passageways 240 and 260 are greatly different, the instrument will indicate that the test is invalid.

The instrument 400 determines how much blood enters the passageways 240 and 260 by diode arrays within instrument 400.

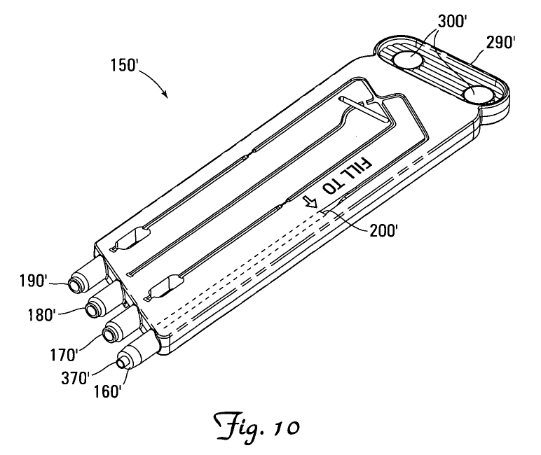

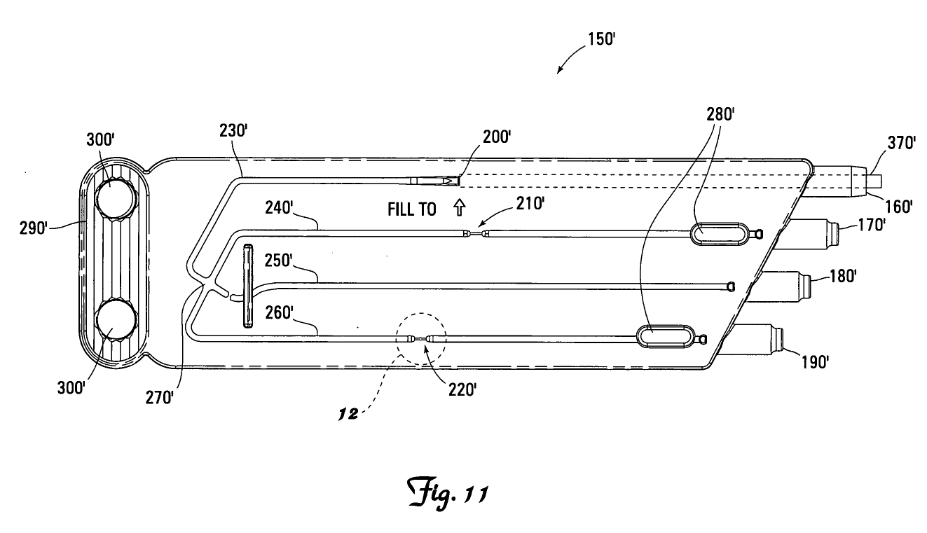

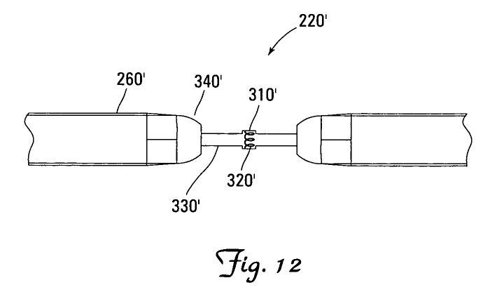

Another example of a device for monitoring platelet function of the invention is shown in FIGS. 10 and 11. Cartridge 150′ has capillary tube inlet 160′ that can receive a capillary tube which is bonded in to capillary tube port 200′. Conduit 230′ leads from capillary tube port 200′ to junction 270′ and junction 270′ splits into passageways 240′, 260′. Passageways 240′, 260′ include overflow wells 280′. Conduit 250′ and passageways 240′, 260′ lead to ports 180′, 170′, 190′, respectively. Although conduit 250′ and port 180′ are retained in the current cartridge they have no present function because conduit 250′ does not connect to junction 270′. Passageways 240′ and 260′ include platelet gel formation portions 210′ and 220′, which include constrictions 340′, narrower conduits 330′, spring housing 310′, and springs 320′. See FIG. 12. Cartridge 150′ includes tab 290′ and recesses 300′.

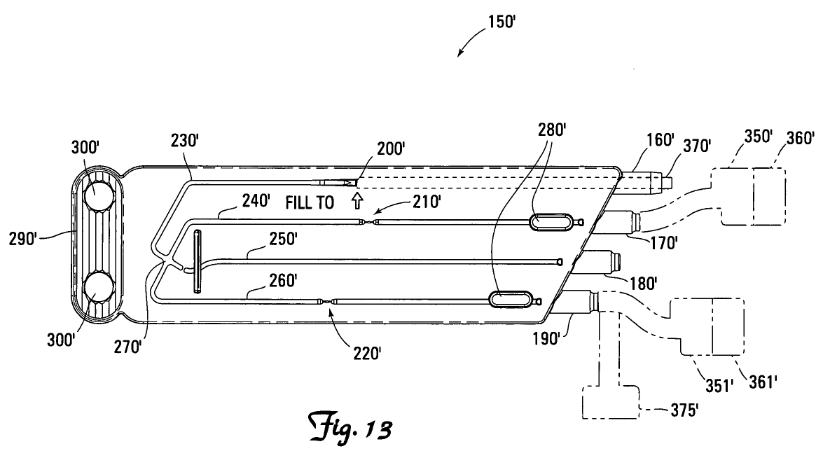

In use, the cartridge is secured in instrument 400′ and preheated for four minutes at 30C. See FIGS. 6 and 7. The cartridge is then removed from the instrument 400 and 40 μl of blood taken from a subject by a pinprick is allowed to wick into capillary tube 370′, which is already bonded to the capillary tube port 200′. See FIG. 13. The cartridge 150′ is then placed back in the instrument 400. When the cartridge is placed back into the instrument the solenoid valve 375′ is open to ensure that blood is not displaced out of the end of the capillary. The instrument 400 is maintained at 30C. In a preferred embodiment, the cartridge 150′ has a bar code that is read by the instrument 400. The instrument 400 will reject the cartridge 150′ if the cartridge has been out of the instrument for more than two minutes after the preheating step or if the cartridge is not the one which was previously warmed.

Bidirectional pumps 350′, 351′ are attached to ports 170′, 190′, respectively. The bidirectional pumps 350′, 351′ are coupled to pressure transducers 360′, 361′ to measure the pressure in passageways 240′, 260′. Pumps 350′ and 351′ can be diaphragm pumps driven by a stepper motor. A solenoid valve 375′ is attached to either the line connecting pump 350′ to port 170′ (not shown) or the line connecting pump 351′ to port 190′ (shown). Port 180′ is capped.

The cartridge 150′ is filled with blood as follows. Solenoid valve 375′ is closed. Pump 351′ and pump 350′ are started. Blood flows into passageway conduits 240′ and 260′. Approximately 15 to 20 μl of blood is now in each of passageways 240′, 260′. If the pumps are run at the same flow rate, the volume of blood in each channel is the same, but, by changing the flow rates, the amount of blood in each passageway can be different.

Next, the bidirectional pumps 350′, 351′ simultaneously cycle the blood back and forth in the passageways 240′ and 260′ between predetermined points. A gel forms and the pumps 350′, 351′ operate until an end point is reached. The end point can be based on pressure, e.g., when the pressure has doubled, or when the pressure has reached a predetermined value such as 9 mmHg, or alternatively on resistance to flow (pressure divided by velocity), e.g., when the resistance to flow is twice the initial resistance to flow. The time it takes to reach the end point is the platelet reactivity time.

The instrument 400 will report the average of the platelet reactivity times determined from the blood in passageways 240′ and 260′. Typical platelet reactivity times range from 10 to 400 seconds. If the two platelet reactivity times determined from the blood in passageways 240′ and 260′ are greatly different, the instrument will indicate that the test is invalid.

The instrument 400 can determine how much blood has entered the passageways 240′ and 260′ by using diode arrays within instrument 400 to measure the length of each blood slug.

The following examples serve to illustrate the present invention and are not intended to limit its scope. The data presented below were generated by the cartridge and method described above in connection with FIGS. 2 to 7 and using a spring made of passivated stainless steel wire with a nominal wire diameter of 0.0015 inch (0.0038 cm), an outside spring diameter of 0.010 inch (0.025 cm) and a length of 0.016 to 0.018 inch (0.041 to 0.046 cm). The spring had 4.5 total coils, 2.5 active coils, and a nominal pitch of 0.0045 inch (0.012 cm) and was placed in a 0.015 inch (0.038 cm) spring recess length.

PRT Versus Collagen Aggregometry

A test cartridge with a single passageway of the same construction as described above but with only one passageway was used in this experiment. The blood was pumped back and forth in the passageway using a constant pressure pump and applying the pressure to alternating ends of the passageway using a system of solenoid valves. For people who have not taken aspirin, the PRT test concludes in less than 100 seconds. After taking aspirin, the PRT increases for most volunteers to greater than 150 seconds. In our latest human volunteer study, 5 volunteers participated and were tested before taking aspirin, one hour after taking aspirin, and then asked them to take an aspirin a day for one week. The volunteers were tested again on days 4 and 7. The PRT tests were run in duplicate and the times were averaged and the results compared to conventional whole blood aggregometry using collagen as the agonist. Collagen was chosen because response to collagen has been shown to differ between people who respond to aspirin and those who do not. In a pilot study with just three volunteers it was shown that the most appropriate concentration of collagen was 2 μg/ml, so this is the concentration that was used. The test was cut off at 100 seconds, so that any PRT which might have run longer than 100 sec is reported as 100 sec.

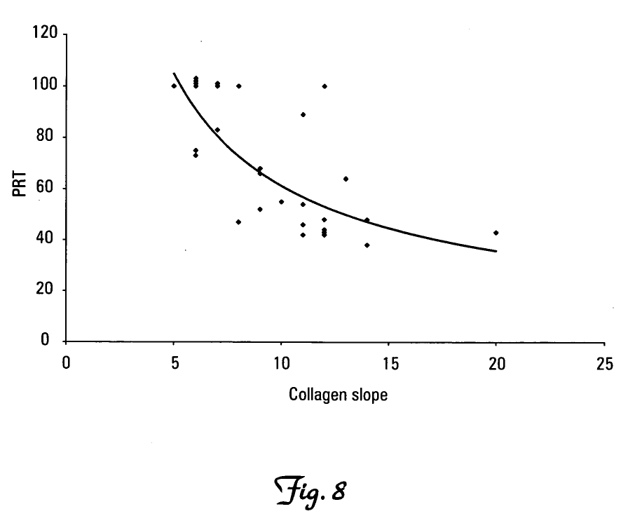

All the data collected from the pilot study and the volunteer study are shown in FIG. 8 (PRT versus collagen), together with a power law trendline. Although this is a small study this is a very significant result because it shows that a 2 minute test on a fingerprick sample of blood correlates with a conventional aggregometry result on a venipuncture sample. In addition, this aggregometry test is one of those used in the literature to identify patients who respond to aspirin. Kawasaki, et al., “Increased Platelet Sensitivity to Collagen in Individuals Resistant to Low-Dose Aspirin,” Stroke, 31(3):591-5 (2000).

PRT Versus Urine 11-Dehydrothromboxane B₂

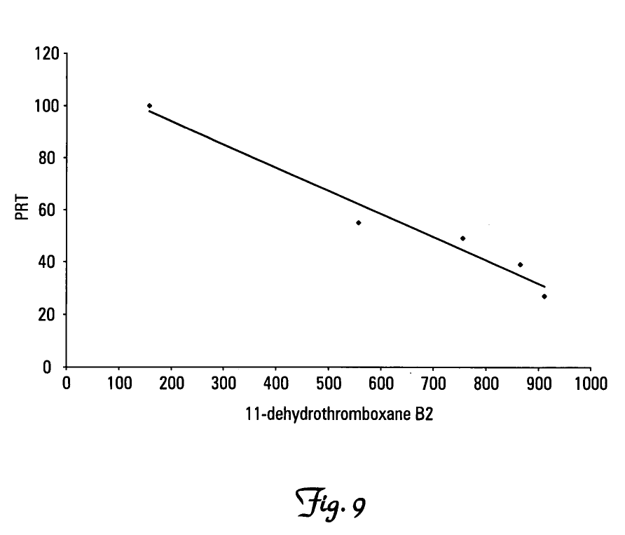

In an earlier study we compared results from the PRT to those obtained by sending samples to an outside reference laboratory to determine levels of thromboxane in the urine. Eikelboom et al., “Aspirin-resistant Thromboxane Biosynthesis and the Risk of Myocardial Infarction, Stroke, or Cardiovascular Death in Patients at High Risk for Cardiovascular Events,” Circulation, 105(14):1650-5 (2002) reported results from a study of 976 patients all taking aspirin in which he measured levels of 11-dehydrothromboxane B₂ which is a metabolite of thromboxane A₂, a biochemical which platelets produce when they are activated. During 5 years of follow-up, those patients having higher levels of this marker also had higher incidence of cardiovascular events including stroke, MI and death from cardiovascular causes. FIG. 9 compares the average PRT results for 5 different donors with the measured levels of the thromboxane metabolite. Once again PRT values greater than 100 sec have been reported as 100 sec. A straight line fit to the data is shown. As can be seen in FIG. 9, PRT correlates with a test which has been shown to predict clinical outcome. Thus, PRT can predict clinical outcome

The above descriptions are provided for the purpose of describing embodiments of the invention and are not intended to limit the scope of the invention in any way. It will be apparent to those skilled in the art that various modifications and variations can be made in the method and device for monitoring platelet function without departing from the spirit or scope of the invention. Thus, it is intended that the present invention cover the modifications and variations of this invention provided they come within the scope of the appended claims and their equivalents.

What is claimed is:

1. A method of monitoring platelet function comprising: passing blood removed from a mammal through a passageway comprising a spring to contact the spring to generate a platelet mass in the passageway; and monitoring the flow or composition of the blood in the passageway to detect formation of the platelet mass.

2. The method of claim 1, wherein the amount of time to form a platelet mass is measured.

3. The method of claim 1, wherein the spring is made of beryllium copper, gold-plated beryllium copper, or stainless steel.

4. The method of claim 1, wherein the spring is made of passivated stainless steel.

5. The method of claim 1, wherein the spring is mounted transversely to the passageway.

6. The method of claim 1, wherein the flow of the blood in the passageway is monitored.

7. The method of claim 6, wherein the flow is monitored by monitoring the pressure of the blood in the passageway.

8. The method of claim 10, wherein the pressure is monitored with a pressure transducer.

9. The method of claim 6, wherein the flow is monitored optically.

10. The method of claim 9, wherein the flow is monitored with a light-emitting diode and a light detector.

11. The method of claim 1, wherein the composition of the blood in the passageway is monitored.

12. The method of claim 1, wherein the passageway and blood do not comprise an added anti-coagulant.

13. The method of claim 1, wherein the passageway does not comprise an added biological agent that activates platelets.

14. The method of claim 1, wherein the blood does not comprise an added biological agent that activates platelets.

15. The method of claim 1, wherein the passageway and blood do not comprise an added chemical agent that activates platelets.

16. The method of claim 1, wherein no biological or chemical agents are added to the removed blood.

17. The method of claim 1, wherein less than 0.4 ml of blood is removed from the body of the mammal.

18. The method of claim 1, wherein less than 20 μl of blood passes through the passageway.

19. The method of claim 1, wherein the blood passes bidirectionally through the passageway.

20. The method of claim 1, wherein the blood is whole blood.

21. The method of claim 1, wherein the blood is fractionated blood.

22. The method of claim 1, wherein the mammal is treated with an anti-platelet agent.

23. The method of claim 22, wherein the anti-platelet agent comprises a cyclooxygenase inhibitor, an ADP inhibitor, a GPIIbIIIa inhibitor, or a combination thereof.

24. The method of claim 1, wherein the platelet mass that forms is substantially depleted in fibrin in comparison to a normal clot.

25. A method of monitoring platelet function comprising: passing blood removed from a mammal through two or more passageways, the two or more passageways comprising an obstruction or irregularity, to contact the obstruction or the wall of the passageways at the irregularity, to generate a platelet mass in the two or more passageways; and monitoring the flow or composition of the blood in the passageways to detect formation of the platelet masses.

26. The method of claim 25, wherein the amount of time to form a platelet mass is measured.

27. The method of claim 25, wherein the formations of the platelet masses in the two or more passageways are detected simultaneously.

28. The method of claim 25, wherein the two or more passageways comprise an obstruction selected from a spring, wire form, metal screen, woven cloth, sheet metal with an orifice, sheet metal forms, polymeric fibers, natural fibers, cellulose fibers, metal wires, suture strands, laser etched or molded plastic formations, glass formations or glass beads.

29. The method of claim 28, wherein the obstruction is a spring.

30. The method of claim 29, wherein the spring is made of beryllium copper, gold-plated beryllium copper, or stainless steel.

31. The method of claim 29, wherein the spring is made of passivated stainless steel.

32. The method of claim 29, wherein the spring is mounted transversely to the passageway.

33. The method of claim 25, wherein the flow of the blood in the two or more passageways is monitored.

34. The method of claim 33, wherein the flow is monitored by monitoring the pressure of the blood in the two or more passageways.

35. The method of claim 34, wherein the pressure is monitored with a pressure transducer.

36. The method of claim 33, wherein the flow is monitored optically.

37. The method of claim 36, wherein the flow is monitored with a light-emitting diode and a light detector.

38. The method of claim 25, wherein the composition of the blood in the two or more passageways is monitored.

39. The method of claim 25, wherein the passageways and blood do not comprise an added anti-coagulant.

40. The method of claim 25, wherein the passageways do not comprise an added biological agent that activates platelets.

41. The method of claim 25, wherein the blood does not comprise an added biological agent that activates platelets.

42. The method of claim 25, wherein the passageways and blood do not comprise an added chemical agent that activates platelets.

43. The method of claim 25, wherein no biological or chemical agents are added to the removed blood.

44. The method of claim 25, wherein less than 0.4 ml of blood is removed from the body of the mammal.

45. The method of claim 25, wherein less than 50 μl of blood passes through the two or more passageways.

46. The method of claim 25, wherein the blood passes bidirectionally through the two or more passageways.

47. The method of claim 25, wherein the blood is whole blood.

48. The method of claim 25, wherein the blood is fractionated blood.

49. The method of claim 25, wherein the mammal is treated with an anti-platelet agent.

50. The method of claim 49, wherein the anti-platelet agent comprises a cyclooxygenase inhibitor, an ADP inhibitor, a GPIIbIIIa inhibitor, or a combination thereof.

51. The method of claim 25, wherein the platelet masses that form are substantially depleted in fibrin in comparison to a normal clot.

52. A device for monitoring platelet function, comprising: a fluid-tight material forming a passageway; a pump functionally linked to the passageway for pumping blood through the passageway; a spring within the passageway, arranged such that when blood is pumped through the passageway to contact the spring, a platelet mass forms on or near the spring; and a detector for detecting the flow of blood through the passageway to detect formation of the platelet mass.

53. A device for monitoring platelet function, comprising: a fluid-tight material forming two or more passageways; two or more pumps functionally linked to the passageways for pumping blood through the passageways; wherein the two or more passageways comprise an obstruction or irregularity arranged such that when blood is pumped through the passageways to contact the obstruction or wall of the passageways at the irregularity, a platelet mass forms on the wall of the passageways at or near the irregularity or at or near the obstruction; and a detector for detecting the flow of blood through the passageways to detect formation of the platelet masses.

54. A device for monitoring platelet function, comprising: a fluid-tight material forming two or more passageways; two or more pumps functionally linked to the passageways for pumping blood through the passageways; wherein the two or more passageways comprise an obstruction or irregularity arranged such that when blood is pumped through the passageways to contact the obstruction or wall of the passageways at the irregularity, a platelet mass forms on the wall of the passageways at or near the irregularity or at or near the obstruction; and a detector for detecting the composition of blood in the passageways to detect formation of the platelet masses.

55. An article for use in a device for monitoring platelet function, the article comprising: a fluid-tight material forming a passageway; and a spring in the passageway, arranged such that when blood is pumped through the passageway to contact the spring, a platelet mass forms on or near the spring.

56. The article of claim 55, wherein the spring is made of beryllium copper, gold-plated beryllium copper, or stainless steel.

57. The article of claim 55, wherein the spring is made of passivated stainless steel.

58. The article of claim 55, wherein the spring is mounted transversely to the passageway.

59. The article of claim 55, wherein the length of the spring is from 0.025 cm to 0.15 cm.

60. An article for use in a device for monitoring platelet function, comprising: a fluid-tight material forming two or more passageways; wherein two or more passageways comprise an obstruction or irregularity arranged such that when blood is pumped through the two or more passageways to contact the obstruction or the wall of the passageway at the irregularity, a platelet mass forms at or near the obstruction or on the wall of the passageway at or near the irregularity.

61. The article of claim 60, wherein the two or more passageways comprise an obstruction selected from a spring, wire form, metal screen, woven cloth, sheet metal with an orifice, sheet metal forms, polymeric fibers, natural fibers, cellulose fibers, metal wires, suture strands, laser etched or molded plastic formations, glass formations or glass beads.

62. The article of claim 60, wherein the obstruction is a spring.

63. The article of claim 62, wherein the spring is made of beryllium copper, gold-plated beryllium copper, or stainless steel.

64. The article of claim 62, wherein the spring is made of passivated stainless steel.

65. The article of claim 62, wherein the spring is mounted transversely to the passageway.

66. The article of claim 62, wherein the length of the spring is from 0.025 cm to 0.15 cm.
